(19) United States
(12) Patent Application Publication
(10) Pub. No.: US 2020/0397756 A1
(43) Pub. Date: Dec. 24, 2020

(54) A STABLE PHARMACEUTICAL COMPOSITION OF POORLY SOLUBLE NONSTEROIDAL ANTIANDROGENS

(71) Applicant: KASHIV BIOSCIENCES, LLC, Bridgewater, NJ (US)

(72) Inventors: Parva Yogeshchandra PUROHIT, Ahmedabad (IN); Paras Rasiklal VASANANI, Ahmedabad (IN); Riteshkumar Baldevbhai GURJAR, Anand (IN); Nisha Ganapatbhai PATEL, Ahmedabad (IN)

(21) Appl. No.: 16/968,422

(22) PCT Filed: Feb. 8, 2019
(86) PCT No.: PCT/IB2019/051028

§ 371 Date: Aug. 7, 2020

(30) Foreign Application Priority Data
Feb. 9, 2018 (IN) IN201821005042

(51) Int. Cl. A61K 31/4166; A61K 47/38; A61K 47/26; A61K 9/48; A61K 9/20

(52) U.S. Cl. CPC A61K 31/4166; A61K 47/38; A61K 47/26; A61K 9/2027; A61K 9/4858; A61K 9/2036; A61K 9/2054; A61K 9/4866

(57) ABSTRACT

The present invention relates to stable pharmaceutical composition of poorly soluble nonsteroidal antiandrogen drug substances suitable for administration for the treatment of prostate cancer. The present invention particularly relates to stable pharmaceutical composition of poorly soluble nonsteroidal antiandrogen (NSAA) drug substances for desirable pharmacokinetic and better patient compliance.

### FIELD OF INVENTION

The present invention relates to stable pharmaceutical composition of poorly soluble nonsteroidal antiandrogen drug substances suitable for administration for the treatment of prostate cancer. The present invention particularly relates to stable pharmaceutical composition of poorly soluble nonsteroidal antiandrogen (NSAA) drug substances for desirable pharmacokinetic and better patient compliance.

### BACKGROUND OF INVENTION

Low aqueous soluble pharmaceutical drug substances are challenging to formulate because of less dissolution property which ultimately leads to less bioavailability.

Enzalutamide (FIG. 1) and apalutamide (FIG. 2) are small molecules with no ionisable groups at biologically relevant pH, therefore, solubility of both is not affected by pH over the physiological range. Enzalutamide and apalutamide exhibit limited aqueous solubility (≤2.0 μg/mL at relevant pH range) and high permeability properties. Due to its low solubility and high permeability, enzalutamide & apalutamide are considered a Biopharmaceutics Classification System (BC S) class 2 drug substances. Apalutamide is a molecule which is very much similar to enzalutamide. Enzalutamide and apalutamide are sparingly soluble in absolute ethanol, and practically insoluble in water between pH 1 to 11.

The chemical name of enzalutamide is 4-{3-[4-cyano-3-(trifluoromethyl)phenyl]-5,5-dimethyl-4-oxo-2-sulfanylideneimidazolidin-1-yl}-2-fluoro-N-methylbenzamide and has the following structure:

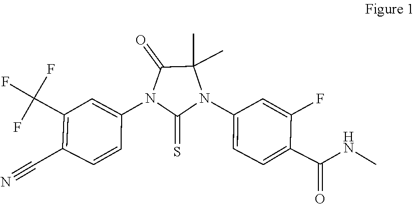 [SMILES: CNC(=O)c1ccc(N2C(=S)N(c3ccc(C#N)c(C(F)(F)F)c3)C(=O)C2(C)C)cc1F]

The chemical name of apalutamide is 4-(7-(6-Cyano-5-(trifluoromethyl)pyridin-3-yl)-8-oxo-6-thioxo-5,7-diazaspiro(3.4)octan-5-yl)-2-fluoro-N-methylbenzamide and has the following structure:

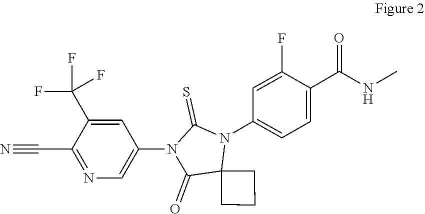

Both enzalutamide and apalutamide are nonsteroidal antagonists. Enzalutamide acts on multiple steps in the androgen receptor signaling pathway. Enzalutamide competitively inhibit androgen binding to the androgen receptor (AR), inhibit AR nuclear translocation, and inhibit AR interaction with DNA. Enzalutamide is used as an agent for treating castration-resistant prostate cancer (CRPC).

The commercial product of enzalutamide (brand name XTANDI®) is an oral liquid-filled soft capsule as enzalutamide fully dissolved in caprylocaproyl polyoxylglycerides, approximately 20 mm×9 mm. The recommended dose is 160 mg enzalutamide (four 40 mg capsules) as a single oral daily dose with or without food. The capsules have a diameter comparable with size 00 capsules which are the largest capsules in use for humans. The capsule should be swallowed whole with water. Patients should not chew or open the capsules. Thus, because commercial available product XTANDI® is having issues of big unit capsule size, there are problems of difficulty in swallowing, which results in situation like dysphagia. This situation ultimately results in very poor patient compliance.

Apalutamide has been approved by USFDA under brand name ERLEADA®. This drug is similar in chemical structure and mechanism to enzalutamide and does not exhibit agonist properties. It is approved for the treatment of patients with non-metastatic castration-resistant prostate cancer. ERLEADA® is approved as 60 mg tablet dosage form and it needs to be administered orally as four tablets once daily. Total daily dose of ERLEADA® is 240 mg for the treatment of patients with non-metastatic castration-resistant prostate cancer.

Grahek R et al., WO2015118015 discloses preparation of nanosuspension composition of enzalutamide using surfactant or polymer as suspension stabilizer. It further describes insufficient effects on enzalutamide dissolution despite of particle size reduction by wet milling in presence of suspension stabilizer.

Lorenz D A et al. US20140179749, Schwarz F et al. US20170216211 and Grahek R et al., WO2015118015 disclose solid dispersion pharmaceutical composition of enzalutamide using polymers.

There is always difficulty in making soluble dosage form with acceptable size for oral administration due to poor solubility of drug in lipophilic oils or polymeric carriers. Above mentioned NSAA drug substances are also having similar kind of chemical property which forms difficulties in developing proper dosage form and ultimately leads to poor patient compliance.

Therefore, there is unmet need to provide pharmaceutical dosage form of poorly soluble NSAA drug substances with smaller size unit dose composition which can be easily swallow by patients and/or having less number of units per daily recommended dose which ultimately leads to better patient compliance. At the same time, there is a need for single unit dosage form of poorly soluble NSAA drug substances which can incorporate higher amount of drug with improved dissolution and bioavailability. Additionally, there is need to reduce the necessary daily dose of poorly soluble NSAA drug substances.

### SUMMARY OF THE INVENTION

The present invention provides stable pharmaceutical composition containing poorly soluble nonsteroidal antiandrogen (NSAA) drug substances. The present invention relates to pharmaceutical dosage form of poorly soluble NSAA drug substances with improved dissolution, bioavailability and better patient compliance.

One aspect of present invention includes stable nanoparticulate pharmaceutical composition of poorly soluble NSAA drug substance. In another aspect of present invention, stable nanoparticulate pharmaceutical composition includes nanosuspension or solid lipid nanoparticulate system. In one another aspect of present invention, stable pharmaceutical composition includes solid dispersion.

In another aspect of the present invention, a stabilized nanosuspension comprise an liquid dispersion medium comprising a surfactant and/or a polymer as stabilizers, wherein particles of a drug substances are suspended within said liquid dispersion medium and optionally combining a de-agglomeration agent with said liquid dispersion medium.

In another aspect of the present invention, a process for preparing stable pharmaceutical nanosuspension composition of poorly soluble NSAA drug substance is provided.

One another aspect of the present invention includes, stable solid lipid nanoparticulate pharmaceutical composition of poorly soluble NSAA drug substance. In another aspect of the present invention, a process for preparing stable pharmaceutical solid lipid nanoparticulate composition of poorly soluble NSAA drug substance is provided.

In another aspect of the present invention, a stabilized solid lipid nanoparticulate composition comprises: i) at least one NSAA drug substance; ii) at least one lipid; and iii) at least one surfactant.

In one another aspect, the present invention provides a solid dispersion containing a poorly soluble NSAA drug substance and one or more pharmaceutically acceptable excipients including but not limited to polymer, surfactant and a combination thereof. In one another aspect, the present invention provides process for preparation of a solid dispersion containing a poorly soluble NSAA drug substance.

In one another aspect, prepared nanosuspension or solid lipid nanoparticulate composition or solid dispersion composition of the present invention can also be used to prepare pharmaceutical dosage forms like minicapsule, capsule, tablet, minitablet, suspension, solution, chewable tablet, orally disintegrating tablet, dispersible tablet, granule, sprinkle, pellet, bead, powder, dry powder for suspension or sachet dosage form etc.

### DETAILED DESCRIPTION OF INVENTION

The stable pharmaceutical compositions of a poorly soluble NSAA drug substance have been developed in response to the present state of the art, and in particular, in response to the problems and unmet needs in the art that have not been fully solved.

As used herein, unless otherwise indicated, the term “poorly soluble” refers to slightly soluble or very slightly soluble drug substance and it requires from about 100 or more parts of solvent for one part of solute and wherein the poorly soluble means the solubilisation of drug substances becomes the rate limiting step for achieving desired bioavailability of such drug substances. It also refers to a drug substance which has a solubility of less than 10 mg/ml, and preferably less than about 5 mg/ml, and more preferably less than about 1.0 mg/ml in aqueous media.

As used herein, unless otherwise indicated, the term “nonsteroidal antiandrogen” (NSAA) drug substance refers to enzalutamide or apalutamide and also other compounds with similar androgen receptor antagonist properties and having similar physicochemical properties and it may include pharmaceutically acceptable derivative, pro-drug, salt, ester, polymorph, or isomer of that recited NSAA drug substance.

As used herein, unless otherwise indicated, the term “active ingredient” refers to one or more NSAA drug substance or combination thereof and it may include pharmaceutically acceptable derivative, pro-drug, salt, ester, polymorph, or isomer of that recited NSAA drug substance.

As used herein, unless otherwise indicated, the terms “about” and “approximately” should be understood to mean within an acceptable error range for the particular value as determined by one of ordinary skill in the art, which will depend in part on how the value is measured or determined, i.e., the limitations of the measurement system. For example, “about” can mean a range of up to 20%, preferably up to 10%, more preferably up to 5%, and more preferably still up to 1% of a given value.

As used herein, unless otherwise indicated, the term “stable” is refers to composition that retains its properties without loss of potency of the active and maintains its physical characteristics over time with minimal degradation.

As used herein, unless otherwise indicated, the terms “nanoparticles”, “nanoparticulate” or “nanosized particles”, which are used interchangeably herein, refer to particles which exhibit a mean particles size that is smaller than about 1000 nm in every dimension. Preferably, “nanoparticles”, “nanoparticulate” or “nanosized particles” exhibit a mean particle size smaller than about 500 nm in every dimension. More preferably, it refers to particles which exhibit a mean particle size smaller than about 200 nm in every dimension.

As used herein, unless otherwise indicated, the term “nanosuspension” can be defined as a colloidal dispersion of nanosized drug particles.

As used herein, unless otherwise indicated, the term “solid dispersion” refers to the dispersion of one or more active ingredients in an inert carrier or matrix at solid state prepared by the spraying of drug dispersion on carrier or matrix, simple wet granulation using drug dispersion, melting, solvent, or melting-solvent methods or a like thereof.

As used herein, unless otherwise indicated, the term “surfactant” used herein is, as generally understood by persons skilled in the art, a substance which can lower the surface tension or interfacial tension between two liquids or between a liquid and a solid. Preferably, the term “surfactant” used herein means a substance capable of acting as wetting agent, as emulsifier, as detergent, and as dispersant. The general function of a substance being a surfactant may be typically known in advance by a person skilled in the art.

The present invention relates to stable pharmaceutical dosage form of a poorly soluble NSAA drug substance with improved dissolution and bioavailability with better patient compliance. The present invention also relates to a pharmaceutical composition of poorly soluble NSAA drug substances which can be easily swallow by patients and/or having less number of units per daily recommended dose which ultimately leads to better patient compliance. Further, the present invention also relates to stable pharmaceutical composition of poorly soluble NSAA drug substance in form of single unit dosage form which can incorporate higher amount of drug with improved dissolution and bioavailability. Additionally, it also covers stable pharmaceutical composition with reduced daily dose of poorly soluble NSAA drug substances compare to current recommended doses. According to present invention, in order to achieve above mentioned characteristics for poorly soluble NSAA drug substances, there are broadly two pharmaceutical approaches, which include first, composition containing nanoparticulate system and secondly, composition containing solid dispersion.

In one aspect of present invention, the stable pharmaceutical composition comprises poorly soluble NSAA drug substance in nanoparticulate form.

Further, in one aspect of present invention, the stable pharmaceutical composition comprises a nanosuspension of a poorly soluble NSAA drug substance. Moreover, the present invention relates to stable pharmaceutical composition comprising a nanosuspension of poorly soluble NSAA drug substances and one or more pharmaceutically acceptable excipients or combination thereof. Further, the present invention relates to a process of preparing a stable pharmaceutical composition comprising a nanosuspension of poorly soluble NSAA drug substances.

In one another aspect, the present invention provides a nanosuspension composition of a poorly soluble NSAA drug substance and one or more pharmaceutically acceptable excipients containing liquid dispersion medium, which includes but not limited to surfactant and/or a polymer as stabilizer and combination thereof. Further, according to one of the embodiment of present invention, nanosuspension composition of poorly soluble NSAA drug substance may contain de-agglomerating agent and other additives thereof.

According to one of the embodiment of the present invention, the techniques for preparing nanosuspension formulation may include bottom-up technologies and top-down technologies. In one another embodiment of the present invention, bottom-up technologies may include but not limited to solvent precipitation, controlled evaporation or other similar techniques. In one another embodiment of the present invention, top-down techniques for preparation of nanosuspension composition may include but not limited to milling, high-pressure homogenization or other similar techniques. Additionally, one of the embodiments of the present invention includes the preparation for nanosuspension composition using combination of both bottom-up and top-down approaches. Moreover, according to one of the embodiment, the process for preparing nanosuspension composition of the poorly soluble NSAA drug substances can be one or more techniques known for various nanosuspension preparation techniques by person skilled in the art at time of the present invention. Various techniques for preparing nanosuspension are described in Leone F et al, Expert Opin. Drug Deliv. (2015) 12(10).

Preferable method for preparation of stable pharmaceutical composition of poorly soluble NSAA drug substance using nanosuspension composition includes but not limited to milling process. More preferable, method for preparation of stable pharmaceutical composition of poorly soluble NSAA drug substance using nanosuspension composition includes but not limited to wet milling process.

According to one of the embodiment, suitable mills include, but are not limited to, a ball mill, an attrition mill, a vibratory mill and other media mills such as a sand mill or a bead mill. The grinding media can comprise particles, preferably spherical in shape, such as beads. Alternatively, the grinding media can comprise particles having a core with a coating of the polymeric resin adhered thereto. Acceptable grinding media for the practice of the present invention include sand, glass beads, metal beads, ceramic beads or similar thereof. Preferred glass beads include barium titanate, soda lime, borosilicate or similar thereof. Preferred metal beads include carbon steel, stainless steel and tungsten carbide or similar thereof. Preferred ceramic beads include yttrium toughened zirconium oxide, zirconium silicate, and alumina. The more preferable grinding media for the purpose of the invention is yttrium toughened zirconium oxide.

In one another aspect of present invention, the nanosuspension of poorly soluble NSAA drug substances may have average sizes less than about 1000 nm, or less than about 800 nm, or less than about 500 nm. In one of the embodiment, preferable particle size of nanosuspension of poorly soluble NSAA drug substance is less than about 800 nm. In one of the embodiment, more preferable particle size of nanosuspension of poorly soluble NSAA drug substance is less than about 500 nm.

In one embodiment of present invention, the stable pharmaceutical dosage form is liquid drug dispersion further contains non-limiting examples of excipients such as stabilizing agents, surfactants, de-agglomerating agents, antioxidants or a like thereof.

In another embodiment liquid drug dispersion is further converted to solid dosage forms like powder, granules, tablet, capsule, sachet or a similar thereof by one of the technique like freeze drying, spraying of drug dispersion on inert bed, wet granulation, solvent evaporation or a like thereof.

In a further embodiment, the present invention relates to a stable nanoparticulate pharmaceutical composition of poorly soluble NSAA drug substance, wherein nanoparticulate composition can be particulate made of an inert solid support coated with the nanosuspension. In a further embodiment, the said such coated particulate may further formulated in form of powder, granules, sachet, capsule or compressed into tablet or a like similar dosage form.

According to another aspect of this present invention, the stable pharmaceutical composition of poorly soluble NSAA drug substance may comprises solid lipid nanoparticulate composition.

In another aspect of the present invention said solid lipid nanoparticles may include: a) at least one poorly soluble NSAA drug substance; b) at least one lipid; and c) at least one surfactant. Optionally, the said solid lipid nanoparticle composition may also include other additives like co-surfactants, pH buffering agents or combinations thereof.

In one aspect of the present invention a process for preparing solid lipid nanoparticles (SLN) of poorly soluble NSAA drug substance is provided. According to one of the embodiment, the process for preparation of SLNs of poorly soluble NSAA drug substances includes but not limited to high energy approaches, low energy approaches, approaches using organic solvents and combinations thereof. High energy approaches may include high-pressure homogenization technique, hot homogenization technique, cold homogenization technique, and high shear homogenization and/or ultrasonication technique. Low energy approaches may microemulsion technique, membrane contactor technique, phase inversion temperature technique, coacervation technique, double emulsion technique, and microemulsion cooling technique. Approaches using organic solvents include emulsification-solvent evaporation technique, emulsification solvent diffusion technique, solvent injection technique, supercritical fluid technique, particle from gas saturated solution technique and gas assisted melting atomisation technique.

According to one of the embodiment of present invention, the preferable process for preparation of SLNs of poorly soluble NSAA drug substances are high shear homogenization, hot homogenization, cold homogenization, ultra sonication or high speed homogenization. Moreover, according to one of the embodiment, the process for preparing SLNs of the poorly soluble NSAA drug substances can be one or more the techniques known for the various SLNs methods by person skilled in the art at time of the present invention. Various techniques for preparing solid lipid nanosuspension are described in Ganeshan et al., Sustainable Chemistry and Pharmacy 6 (2017) 37-56.

Preferable method for preparation of stable pharmaceutical SLN composition of poorly soluble NSAA drug substance includes but not limited to high pressure homogenization. More preferable, method for preparation of stable pharmaceutical SLN composition of poorly soluble NSAA drug substance includes but not limited to hot homogenization technique.

In one of embodiment, the process for preparation of SLNs may include:
a. the lipid and drug are melted above the melting temperature of the lipid;
b. a surfactant solution is heated and added to the drug-lipid mixture at the same temperature;
c. combined surfactant and drug-lipid mixture is converted to a hot pre-emulsion using high shear device which results in the formation of a hot oil/water nano-emulsion;
d. the obtained nanoemulsion is cooled down to room temperature where the lipid recrystallizes and leads to the formation of nanoparticles.

In still another embodiment the formed nanoparticles containing NSAA drug substance is further formulated in form of sachet, a capsule or compressed into tablet or a like similar dosage form.

In a further embodiment, the present invention relates to a method of treating prostate cancer by administering a stable nanoparticulate pharmaceutical composition of poorly soluble NSAA drug substance, wherein the formed nanoparticles containing NSAA drug substance is further formulated in form of sachet, a capsule or compressed into tablet or a like similar dosage form.

In one another aspect of present invention, the nanoparticulate system of poorly soluble NSAA drug substances are having average sizes less than about 1000 nm, or less than about 800 nm, or less than about 500 nm. In one of the embodiment, preferable particle size of nanoparticulate system of poorly soluble NSAA drug substance is less than about 800 nm. In one of the embodiment, more preferable particle size of nanoparticulate system of poorly soluble NSAA drug substance is less than about 500 nm.

In another aspect of present invention, the stable pharmaceutical composition comprises solid dispersion of poorly soluble NSAA drug substance.

In a one another aspect, the present invention provides a solid dispersion containing a poorly soluble NSAA drug substance and one or more pharmaceutically acceptable excipients including but not limited to polymer, surfactant and a combination thereof.

According to one of the embodiment of the present invention, the techniques for preparing solid dispersion composition may include melting method, solvent method or melting-solvent method. Preferably, according to one of the embodiment of the present invention, the techniques for preparing solid dispersion composition may include melting method.

Moreover, according to one of the embodiment, the process for preparing solid dispersion of the poorly soluble NSAA drug substances can be one or more techniques known for various solid dispersion methods by person skilled in the art at time of the present invention. Various techniques for preparing solid dispersion are described in Chiou and Riegelman, Journal of Pharmaceutical Sciences, 60, 1281 (1971).

In still another embodiment the formed solid dispersion composition containing NSAA drug substance is further formulated in form of sachet, capsule or compressed into tablet or a like similar dosage form.

In a further embodiment, the present invention relates to a method of treating prostate cancer by administering a stable solid dispersion pharmaceutical composition of poorly soluble NSAA drug substance, wherein the formed solid dispersion composition containing NSAA drug substance is further formulated in form of sachet, capsule or compressed into tablet or a like similar dosage form.

Non-limiting examples of pharmaceutically acceptable excipients are,

### Surfactants:

The surfactants used for the preparation of stable pharmaceutical composition in accordance with the present invention include anionic surfactants, cationic surfactants, non-ionic surfactants, zwitterionic surfactants or combinations thereof. The cationic surfactant includes but not limited to quaternary ammonium compound (e.g. benzalkonium chloride, cetylpyridinium chloride, benzethonium chloride and a like and combinations thereof), alkyl ammonium salt (e.g. alkyl trimethylammonium salts, cetyl trimethylammonium bromide, cetyl trimethylammonium chloride, dimethyldioctadecylammonium chloride, dioctadecyldimethylammonium bromide and a like or combinations thereof), octenidine dihydrochloride and a like or combinations thereof. The anionic surfactant includes but not limited to sodium lauryl sulphate, ammonium lauryl sulphate, sodium lauryl ether sulphate, sodium myreth sulphate, sodium stearate, dioctyl sodium sulfosuccinate, perfluorooctanesulfonate, linear alkylbenzene sulfonates, perfluorobutanesulfonate, alkyl-aryl ether phosphates, alkyl ether phosphates, sodium lauroyl sarcosinate, perfluorononanoate, perfluorooctanoate and a like or combinations thereof. The zwitterionic surfactant includes but not limited to (3-[(3-cholamidopropyl)dimethylammonio]-1-propanesulfonate), cocamidopropyl hydroxysultaine, cocamidopropyl betaine, phospholipids phosphatidylserine, phosphatidylethanolamine, phosphatidylcholine, phosphatidyl choline-enriched lecithin, sphingomyelins, lecithin and a like or combinations thereof. The non-ionic surfactant includes but not limited to polyoxyethylene alkyl ethers (e.g. polyoxyethylene glycol octylphenol ethers, polyoxyethylene glycol alkylphenol ethers and a like thereof), polyoxyethylene sorbitan fatty acid esters, polyoxyethylene stearates, sorbitan fatty acid esters, polyoxyethylene castor oil derivatives, block copolymers of polyethylene glycol, polypropylene glycol, alkyl poly(ethylene oxide), copolymers of poly(ethylene oxide) and poly(propylene oxide), alkyl polyglucosides (e.g. octyl glucoside, decyl maltoside and a like or combinations thereof), fatty alcohols (e.g. cetyl alcohol, oleyl alcohol and a like or combinations thereof), fatty acid amides (e.g. cocamide monoethanolamine, cocamide diethanolamine and a like or combinations thereof), and dodecyl dimethylamine oxide, vitamin E-TPGS, caprylocaproyl macrogolglycerides, alkyl aryl polyether alcohol (e.g. tyloxapol) and a like or combinations thereof. The surfactants may present in an amount of about 0.01% to about 60% by weight of composition, preferably of about 0.1% to about 40% by weight of composition.

### Polymers:

The polymers used for the preparations of stable pharmaceutical composition of poorly soluble NSAA agent include but not limited to cellulosic or non-cellulosic polymers. The polymers may be ionisable, non-ionisable or neutral in aqueous solution. In some embodiments, polymers are amphiphilic in nature, meaning that the polymer has hydrophobic and hydrophilic portions. The hydrophobic portion may comprise groups such as aliphatic or aromatic hydrocarbon groups. The hydrophilic portion may comprise either ionisable or non-ionisable groups that are capable of hydrogen bonding such as hydroxyls, carboxylic acids, esters, amines or amides. In another embodiment of present invention, the polymers include water soluble or water insoluble polymers. Non-limiting examples of polymers includes hydroxyethyl cellulose, ethylcellulose, hydroxypropyl cellulose, hydroxypropyl methyl cellulose, methyl cellulose, polyvinyl pyrrolidone, polyvinyl alcohol, polyacrylic acid, polyethylene glycol, polyethylene oxide, copovidone, hydroxypropyl methylcellulose acetate succinate, hydroxypropyl methylcellulose K100 LVCR, polyacrylates (Eudragit RSPO, Eudragit RLPO etc.), carboxymethyl cellulose derivatives (carboxymethyl cellulose, carboxymethyl cellulose acetate butyrate, etc.), cellulose phthalate derivatives (hydroxypropylmethyl cellulose phthalate, cellulose acetate phthalate, etc.), cellulose ω-carboxy esters (cellulose acetate adipate propionate, cellulose acetate sub-cellulose acetate suberate, cellulose acetate adipate, cellulose acetate sebacate, etc.), poloxamers, chitosan, agar, pectin, polylysine or gelatine and a like or combinations thereof. Additionally, non-limiting examples of polymers may also include cellulosic polymers, sugars (e.g. lactose or a like thereof), sugar alcohols, inorganic oxides, inorganic salts and metal silicate materials (e.g. aluminosilicates or a like thereof), starch, or a like and combination thereof. The polymers may present in an amount of about 0.01% to about 70% by weight of composition, preferably of about 0.1% to about 40% by weight of composition.

### De-Agglomeration Agents:

The de-agglomeration agents used for preparation of stable pharmaceutical composition can be an anionic or cationic polymer. In still a further embodiment of the invention the anionic polymer is a copolymer of methacrylic acid and an acrylate selected from the group consisting of ethyl acrylate, methacrylate, and methyl methacrylate and a like or combinations thereof. In still another embodiment of the invention the cationic polymer is based on a copolymer of dimethylaminoethyl methacrylate, butyl methacrylate, and methyl methacrylate and a like or combinations thereof. The de-agglomeration agents may present in an amount of about 0.001% to about 20% by weight of composition, preferably of about 0.1% to about 10% by weight of composition.

### Lipids:

The lipids used for preparation of stable pharmaceutical composition of poorly soluble NSAA agent is refers to but not limited to triglycerides (e.g. tristearin, tripalmitin, tristearin and a like or combinations thereof), diglycerides, monoglycerides or combinations thereof (e.g. glycerol behenate, glycerol monostearate, glyceryl palmitostearate and a like or combinations thereof), fatty acids (e.g. myristic acid, stearic acid, palmitic acid, tetradecanoic acid, arachidic acid, behenic acid and a like or combinations thereof), steroids (e.g. cholesterol and a like or combinations thereof), waxes (e.g. carnauba wax, beeswax, cetyl palmitate and a like or combinations thereof) and other lipids (e.g. milglyol 812, paraffin and a like or combinations thereof). The lipids may present in an amount of about 0.01% to about 70% by weight of composition, preferably of about 0.1% to about 40% by weight of composition.

### Antioxidants:

The antioxidants used for preparation of stable pharmaceutical composition of poorly soluble NSAA agent includes but not limited to butylated hydroxyanisole, butylated hydroxytoluene, tetra butyl hydroquinone, gallic acid, propyl gallate, α-tocopherol, ascorbic acid, citric acid, L-cysteine, thioglycolic acid, ascorbic acid, ascorbyl palmitate, monothioglycerol, sodium bisulfite, sodium metabisulfite, sodium sulphite, trisodium citrate, disodium edetate, sodium diethyldithiocarbamate, fumaric acid, malic acid, phosphoric acid, tartaric acid, glutamic acid, oxalic acid and a like or combinations thereof. The antioxidants may present in an amount of about 0.001% to about 20% by weight of composition, preferably of about 0.1% to about 10% by weight of composition.

In some embodiment of the invention, sugars and sugar alcohols may also be used for preparation of nanosuspension composition of poorly soluble NSAA drug substances. In another embodiment of the invention, sugars and sugar alcohols includes but not limited to lactose, sucrose, mannitol and a like or combinations thereof.

In one of the embodiment of the present invention, the poorly soluble NSAA drug substance may administered as a unit stable pharmaceutical composition, for example, containing 0.05 to 1000 mg by weight, preferably 0.5 to 1000 mg by weight, more preferably 5 to 500 mg by weight of drug substance.

In some embodiments of present invention, a stable pharmaceutical composition may comprise, for example, at least about 0.1% by weight or at least about 5% by weight or at least about 20% by weight or at least about 50% by weight or at least about 75% by weight of a NSAA drug substance.

In one of the embodiment of present invention, NSAA drug substance is present in amorphous form. In one another embodiment of present invention, the amorphous form of NSAA drug substance may be present as at least about 30-100% as amorphous form, preferably at least about 50% as amorphous form, more preferably at least about 70% as amorphous form, yet more preferably about 90% as amorphous form. In one more embodiment of the present invention, NSAA drug substance present in amorphous form is sufficiently stable and well suited for use in stable pharmaceutical compositions, which are useful in the treatment of disease, including, but not limited to, prostate cancer.

According to another embodiment of this present invention, a stable pharmaceutical composition can be included within a pharmaceutical composition by use of one or more pharmaceutically acceptable excipients or combinations thereof.

According to another embodiment of this present invention, a stable pharmaceutical composition of poorly soluble NSAA drug substance can be provided in the form of minicapsule, capsule, tablet, minitablet, suspension, solution, chewable tablet, orally disintegrating tablet, dispersible tablet, granule, sprinkle, pellet, bead, powder, dry powder for suspension or sachet dosage form. These forms are well known in the art and are packaged appropriately.

According to another embodiment of this present invention, liquid stable pharmaceutical compositions can prepared by dissolving or suspending poorly soluble NSAA drug substances in a liquid vehicle acceptable for pharmaceutical administration so as to provide the desired dosage.

According to another embodiment of this present invention, a stable pharmaceutical composition can be coated with one or more enteric coatings, seal coatings, film coatings, barrier coatings, or fast disintegrating coatings. Further, the dosage form can be designed for immediate release, controlled release or extended release of poorly soluble NSAA drug substance.

According to another embodiment, a stable pharmaceutical composition of poorly soluble NSAA drug substances may be further formulated according to accepted pharmaceutical practice with one or more pharmaceutically acceptable excipients, wherein one or more pharmaceutically acceptable excipients may include diluents, binders, disintegrants, glidants, lubricants, preservative, stabilizer, flavouring agent, sweetening agent or mixtures thereof.

Non-limiting examples of diluents that may be suitable for use in the present invention for further formulating poorly soluble NSAA drug substances include inorganic phosphates like dibasic calcium phosphate, sugars or sugar analogues and derivatives thereof (e.g. lactose, such as lactose monohydrate or water-free lactose, dextrose, sorbitol, mannitol, saccharose, maltodextrin, isomalt etc.), celluloses (e.g. microcrystalline cellulose etc.) or a like thereof. The diluents may present in an amount of about 1% to about 90% by weight of composition, preferably of about 10% to about 80% by weight of composition, more preferably of about 40% to about 70% by weight of composition.

Non-limiting examples of binders that may be suitable for use in the present invention for further formulating poorly soluble NSAA drug substances include polyvinylpyrrolidone, starch, cellulose derivatives like hydroxypropylmethyl cellulose, sucrose, lactose, xylitol, sorbitol, maltitol, water, alcohol or polyethylene glycol or a like thereof. According to the present invention, preferable binder is lactose, polyvinylpyrrolidone or polyethylene glycol. The binders may present in an amount of about 1% to about 90% by weight of composition, preferably of about 10% to about 70% by weight of composition.

Non-limiting examples of disintegrants that may be suitable for use in the present invention for further formulating poorly soluble NSAA drug substances include sodium starch glycolate, alginates, pregelatinized starch, croscarmellose and cross-linked polyvinylpyrrolidone and a like or combinations thereof. The disintegrants may present in an amount of about 1% to about 10% by weight of composition, preferably of about 4% to about 8% by weight of composition.

Non-limiting examples of glidants that may be suitable for use in the present invention for further formulating poorly soluble NSAA drug substances include silicon dioxide, talc, magnesium stearate or a like thereof. A preferred glidants is talc and magnesium stearate. The preferred amount of glidant is of about 0.1% to about 10% by weight of composition.

Non-limiting examples of lubricants that may be suitable for use in the present invention for further formulating poorly soluble NSAA drug substances include fatty acids or fatty acid derivatives, such as alkali and earth alkali salts of stearic, lauric acid or palmitic acid or a like thereof. A preferred lubricant is magnesium stearate. The preferred amount of lubricants is about 0.1% to about 5% by weight of composition.

Non-limiting examples of a sweetening agent includes but not limited to sucrose, aspartame, lactose or saccharin or a like and combination thereof; a flavouring agent includes but not limited to orange, peppermint, oil of wintergreen or cherry or a like and combinations thereof.

According to another embodiment of this present invention, the dose administered must be carefully adjusted according to age, weight and condition of the patient, as well as the route of administration, dosage form and regimen and the desired result. According to another embodiment of this present invention, dosage forms can be administered to the patient on a regimen of, for example, one, two, three, four, five, six, or other doses per day.

The following examples further describe certain specific aspects and embodiments of the disclosure. However, one skilled in the art will appreciate that the specific methods and results discussed are provided only for purposes of illustration, and should not be construed as limiting the scope of the disclosure in any manner, as many variations thereof are possible without departing from the spirit and scope of the invention.

### Example 1

### Preparation of Enzalutamide Nanosuspension:

a. Suspension preparation for wet-milling:
1) Clear solution of hydroxypropyl methylcellulose, lecithin and sodium lauryl sulfate into purified water is formed.
2) Enzalutamide is slowly added into mixture formed in step (1), until no visible particles are observed on the surface & homogenized using homogenizer.

b. Wet-milling:
1) The suspension as prepared in step (a) is wet milled using zirconium bead as milling media within pearl mill.
2) Wet milled nanosuspension is then collected and continuously mixed at low speed with a portable stirrer.

c. Nanosuspension preparation:
Sodium lauryl sulfate & sucrose added to the nanosuspension prepared in step (b) and mixed.

TABLE 1
Preparation of enzalutamide nanosuspension

| Ingredients | % w/w |
|---|---|
| Enzalutamide | 5-60% |
| Hydroxypropyl methylcellulose | 1-20% |
| Sodium lauryl sulfate | 0.01-5% |
| Lecithin | 0.1-10% |
| Sucrose | 5-50% |
| Purified Water | Q.s. |

### Example 2

### Top Spray Granulation of Nanosuspension:

1) The enzalutamide nanosuspension for top spray granulation is sprayed on lactose monohydrate.
2) After completion of spraying, the intermediate granules are dried until loss of drying of NMT 2.0% is achieved.

### Example 3

### Milling, Blending & Filling:

1) Dried intermediate granules are milled and then blended with crospovidone, magnesium stearate and microcrystalline cellulose for final blending.
2) Final blended composition is filled into capsule.

TABLE 2
Preparation of enzalutamide formulation

| Ingredients | % w/w |
|---|---|
| Enzalutamide Nanosuspension | 20-70% |
| Lactose Monohydrate | 5-60% |
| Crospovidone | 1-10% |
| Microcrystalline Cellulose | 10-80% |
| Magnesium Stearate | 0.1-10% |

### Example 4

| Ingredients | % w/w | Amount (mg) |
|---|---|---|
| Enzalutamide | 10.3 | 160.0 |
| Hydroxypropyl methylcellulose phthalate | 30.9 | 480.0 |
| (HP-55) | | |
| Acetone | q.s. | q.s. |
| Microcrystalline cellulose (PH 101) | 51.5 | 800.0 |
| Silicon dioxide | 4.1 | 64.0 |
| Talc | 3.2 | 50.0 |
| Total | 100 | 1554.0 |

### Manufacturing Process:

1. Hydroxypropyl methylcellulose phthalate (HP-55) was dissolved in acetone under continuous stirring.
2. Enzalutamide is added to solution of step 1 under continuous stirring till completely dissolved.
3. Solvent evaporated from step 2 using solvent evaporation technique at 55° C.
4. Dried powder is mixed with silicon dioxide, talc, microcrystalline cellulose (PH 101) and sifted through sieve.
5. Optionally, final blended composition is compressed in form of tablet.

### Example 5

| Ingredients | % w/w | Amount (mg) |
|---|---|---|
| Enzalutamide | 10.3 | 160.0 |
| Hydrogenated castor oil | 30.9 | 480.0 |
| Methylene chloride | q.s. | q.s. |
| Microcrystalline cellulose (PH 101) | 51.5 | 800.0 |
| Silicon dioxide | 4.1 | 64.0 |
| Talc | 3.2 | 50.0 |
| Total | 100 | 1554.0 |

### Manufacturing Process

1. Hydrogenated Castor Oil dissolved in methylene chloride under continuous stirring.
2. Enzalutamide is added to solution of step 1 under continuous stirring till completely dissolved.
3. Solvent evaporated from step 2 using solvent evaporation technique at 55° C.
4. Dried powder is mixed with silicon dioxide, talc and microcrystalline cellulose (PH 101) and sifted through sieve.
5. Optionally, final blended composition is compressed in form of tablet.

### Example 6

| Ingredients | % w/w | Amount (mg) |
|---|---|---|
| Enzalutamide | 10.3 | 160.0 |
| Hydroxypropyl methylcellulose (K100 | 30.9 | 480.0 |
| LVCR) | | |
| Methylene Chloride | q.s. | q.s. |
| Microcrystalline cellulose (PH 101) | 51.5 | 800.0 |
| Silicon dioxide | 4.1 | 64.0 |
| Talc | 3.2 | 50.0 |
| Total | 100 | 1554.0 |

### Manufacturing Process:

1. Hydroxypropyl methylcellulose (K100 LVCR) dissolved in methylene chloride under continuous stirring.
2. Enzalutamide is added to solution of step 1 under continuous stirring till completely dissolved.
3. Solvent evaporated from step 2 using solvent evaporation technique at 55° C.
4. Dried powder is mixed with silicon dioxide, talc and microcrystalline cellulose (PH 101) and sifted through sieve.
5. Optionally, final blended composition is compressed in form of tablet.

### Example 7

| Ingredients | % w/w | Amount (mg) |
|---|---|---|
| Enzalutamide | 10.3 | 160.0 |
| Eudragit RSPO | 30.9 | 480.0 |
| Acetone | q.s. | q.s. |
| Microcrystalline cellulose (PH 101) | 51.5 | 800.0 |
| Silicon dioxide | 4.1 | 64.0 |
| Talc | 3.2 | 50.0 |
| Total | 100 | 1554.0 |

### Manufacturing Process

1. Eudragit RSPO dissolved in acetone under continuous stirring.
2. Enzalutamide is added to solution of step 1 under continuous stirring till completely dissolved.
3. Solvent evaporated from step 2 using solvent evaporation technique at 55° C.
4. Dried powder is mixed with silicon dioxide, talc and microcrystalline cellulose (PH 101) and sifted through sieve.
5. Optionally, final blended composition is compressed in form of tablet.

### Example 8

| Ingredients | % w/w | Amount (mg) |
|---|---|---|
| Enzalutamide | 10.3 | 160.0 |
| Eudragit RLPO | 30.9 | 480.0 |
| Acetone | q.s. | q.s. |
| Microcrystalline cellulose (PH 101) | 51.5 | 800.0 |
| Silicon dioxide | 4.1 | 64.0 |
| Talc | 3.2 | 50.0 |
| Total | 100 | 1554.0 |

### Manufacturing Process:

1. Eudragit RLPO dissolved in acetone under continuous stirring.
2. Enzalutamide is added to solution of step 1 under continuous stirring till completely dissolved.
3. Solvent evaporated from step 2 using solvent evaporation technique at 55° C.
4. Dried powder is mixed with silicon dioxide, talc and microcrystalline cellulose (PH 101) and sifted through sieve.
5. Optionally, final blended composition is compressed in form of tablet.

### Example 9

| Ingredients | % w/w | Amount (mg) |
|---|---|---|
| Enzalutamide | 10.3 | 160.0 |
| Hydroxypropyl methylcellulose acetate | 30.9 | 480.0 |
| succinate (MG) | | |
| Acetone | q.s. | q.s. |
| Microcrystalline cellulose (PH 101) | 51.5 | 800.0 |
| Silicon dioxide | 4.1 | 64.0 |
| Talc | 3.2 | 50.0 |
| Total | 100 | 1554.0 |

### Manufacturing Process:

1. Hydroxypropyl methylcellulose acetate succinate (MG) dissolved in acetone under continuous stirring.
2. Enzalutamide is added to solution of step 1 under continuous stirring till completely dissolved.
3. Solvent evaporated from step 2 using solvent evaporation technique at 55° C.
4. Dried powder is mixed with silicon dioxide, talc and microcrystalline cellulose (PH 101) and sifted through sieve.
5. Optionally, final blended composition is compressed in form of tablet.

### Example 10

| Ingredients | % w/w | Amount (mg) |
|---|---|---|
| Enzalutamide | 9.1 | 160.0 |
| Hydroxypropyl methylcellulose acetate | 27.4 | 480.0 |
| succinate (MG) | | |
| Acetone | q.s. | q.s. |
| Lactose monohydrate | 55.3 | 967.0 |
| Silicon dioxide | 4.3 | 75.0 |
| Colloidal silicon dioxide | 1.9 | 33.0 |
| Talc | 2.0 | 35.0 |
| Total | 100 | 1750.0 |

### Manufacturing Process:

1. Hydroxypropyl methylcellulose acetate succinate (MG) dissolved in acetone under continuous stirring.
2. Enzalutamide is added to solution of step 1 under continuous stirring till completely dissolved.
3. Solvent evaporated from step 2 using solvent evaporation technique at 55° C.
4. Dried powder is mixed with lactose monohydrate, silicon dioxide, colloidal silicon dioxide and talc and sifted through sieve.
5. Optionally, final blended composition is compressed in form of tablet.

We claim:

1. A stable pharmaceutical composition comprising enzalutamide or pharmaceutically acceptable salts thereof and pharmaceutically acceptable excipients, wherein pharmaceutical composition is a single unit dosage form comprising greater than 80 mg enzalutamide or pharmaceutically acceptable salts thereof.

2. The stable pharmaceutical composition according to claim 1, wherein total amount of enzalutamide or pharmaceutically acceptable salts thereof is about 160 mg.

3. The stable pharmaceutical composition according to claim 2, wherein pharmaceutical composition is in the form of tablet or capsule.

4. The stable pharmaceutical composition according to claim 1, wherein at least 50% of enzalutamide or pharmaceutically acceptable salts thereof is in amorphous form.

5. The stable pharmaceutical composition according to claim 1, wherein enzalutamide or pharmaceutically acceptable salts thereof is present in the form of nanoparticles, nanosuspension, solid lipid nanoparticles or solid dispersion in pharmaceutical composition.

6. The stable pharmaceutical composition according to claim 1, wherein the said pharmaceutical composition is present in the form of capsule, minicapsule, tablet, minitablet, suspension, solution, chewable tablet, orally disintegrating tablet, dispersible tablet, granule, sprinkle, pellet, bead, powder, dry powder for suspension or sachet, or a like thereof.

7. The stable pharmaceutical composition according to claim 1, wherein enzalutamide or pharmaceutically acceptable salts thereof is present in an amount from about 80 mg to less than 160 mg with improved bioavailability compared to XTANDI® (total four 40 mg capsules).

8. The stable pharmaceutical composition according to claim 1, wherein the said pharmaceutical composition contains solid dispersion of enzalutamide or pharmaceutically acceptable salts thereof and at least one polymer.

9. The stable pharmaceutical composition according to claim 8, wherein said pharmaceutical composition contains at least one polymer selected from the group consisting of hydroxyethyl cellulose, ethylcellulose, hydroxypropyl cellulose, hydroxypropyl methyl cellulose, methyl cellulose, polyvinyl pyrrolidone, polyvinyl alcohol, polyacrylic acid, polyethylene glycol, polyethylene oxide, copovidone, hydroxypropyl methylcellulose acetate succinate, hydroxypropyl methylcellulose K100 LVCR, polyacrylates (Eudragit RSPO, Eudragit RLPO etc.), carboxymethyl cellulose derivatives (carboxymethyl cellulose, carboxymethyl cellulose acetate butyrate, etc.), cellulose phthalate derivatives (hydroxypropylmethyl cellulose phthalate, cellulose acetate phthalate, etc.), cellulose ω-carboxy esters (cellulose acetate adipate propionate, cellulose acetate sub-cellulose acetate suberate, cellulose acetate adipate, cellulose acetate sebacate, etc.), poloxamers, chitosan, agar, pectin, polylysine, cellulosic polymers, sugars (e.g. lactose or a like thereof), sugar alcohols, inorganic oxides, inorganic salts and metal silicate materials (e.g. aluminosilicates or a like thereof), starch or gelatine and a like or combinations thereof.

10. A method of treating prostate cancer by administering stable pharmaceutical composition according to claim 2, to a subject in need of such treatment.

11. A stable pharmaceutical composition comprising apalutamide or pharmaceutically acceptable salts thereof and pharmaceutically acceptable excipients, wherein pharmaceutical composition is a single unit dosage form comprising greater than 120 mg apalutamide or pharmaceutically acceptable salts thereof.

12. The stable pharmaceutical composition according to claim 11, wherein total amount of apalutamide or pharmaceutically acceptable salts thereof is about 240 mg.

13. The stable pharmaceutical composition according to claim 12, wherein pharmaceutical composition is in form of tablet or capsule.

14. The stable pharmaceutical composition according to claim 11, wherein at least 50% of apalutamide or pharmaceutically acceptable salts thereof is in amorphous form.

15. The stable pharmaceutical composition according to claim 11, wherein apalutamide or pharmaceutically acceptable salts thereof is present in the form of nanoparticles, nanosuspension, solid lipid nanoparticles or solid dispersion in pharmaceutical composition.

16. The stable pharmaceutical composition according to claim 11, wherein the said pharmaceutical composition is present in the form of capsule, minicapsule, tablet, minitablet, suspension, solution, chewable tablet, orally disintegrating tablet, dispersible tablet, granule, sprinkle, pellet, bead, powder, dry powder for suspension or sachet, or a like thereof.

17. The stable pharmaceutical composition according to claim 11, wherein apalutamide or pharmaceutically acceptable salts thereof is present in an amount from about 120 mg to less than 240 mg with improved bioavailability compared to ERLEADA® (total four 60 mg tablets).

18. The stable pharmaceutical composition according to claim 11, wherein the said pharmaceutical composition contains solid dispersion of apalutamide or pharmaceutically acceptable salts thereof and at least one polymer.

19. The stable pharmaceutical composition according to claim 18, wherein said pharmaceutical composition contains at least one polymer selected from the group consisting of hydroxyethyl cellulose, ethylcellulose, hydroxypropyl cellulose, hydroxypropyl methyl cellulose, methyl cellulose, polyvinyl pyrrolidone, polyvinyl alcohol, polyacrylic acid, polyethylene glycol, polyethylene oxide, copovidone, hydroxypropyl methylcellulose acetate succinate, hydroxypropyl methylcellulose K100 LVCR, polyacrylates (Eudragit RSPO, Eudragit RLPO etc.), carboxymethyl cellulose derivatives (carboxymethyl cellulose, carboxymethyl cellulose acetate butyrate, etc.), cellulose phthalate derivatives (hydroxypropylmethyl cellulose phthalate, cellulose acetate phthalate, etc.), cellulose ω-carboxy esters (cellulose acetate adipate propionate, cellulose acetate sub-cellulose acetate suberate, cellulose acetate adipate, cellulose acetate sebacate, etc.), poloxamers, chitosan, agar, pectin, polylysine, cellulosic polymers, sugars (e.g. lactose or a like thereof), sugar alcohols, inorganic oxides, inorganic salts and metal silicate materials (e.g. aluminosilicates or a like thereof), starch or gelatine and a like or combinations thereof.

20. A method of treating prostate cancer by administering stable pharmaceutical composition according to claim 12, to a subject in need of such treatment.
